(19) United States
(12) Patent Application Publication
(10) Pub. No.: US 2022/0402763 A1
(43) Pub. Date: Dec. 22, 2022

(54) GRAPHENE OXIDE (GO)-BASED COMPOSITE NANOPARTICLE DRUG DELIVERY SYSTEM AND PREPARATION METHOD THEREOF

(71) Applicant: Qufu Normal University, Jining (CN)

(72) Inventors: Ge Yang, Jining (CN); Ruixia Gao, Jining (CN); Chengchuan Che, Jining (CN); Jinfeng Liu, Jining (CN); Zhijin Gong, Jining (CN)

(73) Assignee: Qufu Normal University, Jining (CN)

(21) Appl. No.: 17/807,456

(22) Filed: Jun. 17, 2022

(30) Foreign Application Priority Data
Jun. 17, 2021 (CN) 202110669641.6

(51) Int. Cl. C01B 32/198; B82Y 5/00; B82Y 30/00; B82Y 40/00; A61P 35/00; A61K 47/02

(52) U.S. Cl. CPC C01B 32/198; B82Y 5/00; B82Y 30/00; B82Y 40/00; A61P 35/00; A61K 47/02

(57) ABSTRACT

The present disclosure belongs to the technical field of biomedicine, and in particular relates to a graphene oxide (GO)-based composite nanoparticle drug delivery system for treating cervical cancer and a preparation method thereof. The composite nanoparticle drug delivery system includes an aptamer NH₂-AS1411 (Aptamer NH2-AS1411, APT), monolayer graphene oxide (GO), chitosan oligosaccharide (CO) and γ-polyglutamic acid (γ-PGA).

### TECHNICAL FIELD

The present disclosure belongs to the technical field of biomedicine, and in particular relates to a graphene oxide (GO)-based composite nanoparticle drug delivery system for cervical

### BACKGROUND ART

Photothermal therapy (PTT) is a new method for local treatment of tumors by utilizing the characteristics of certain biological materials that absorb light energy in near-infrared regions and convert it into heat energy. PTT kills tumor cells and has no toxic or side effects on human bodies, and is called a “green therapy” by the international medical community. In recent years, methods for cancer treatment that utilize the characteristics of GO to absorb light energy at 808 nm in a near-infrared region and convert it into heat energy in combination with chemical drugs have received extensive attention. After loading anti-tumor drugs with GO or derivatives thereof into a tumor site, a near-infrared laser irradiation at 808 nm is conducted to generate heat to heat the tumor site, changing the permeability of capillaries and increasing the concentration of drugs in blood. The heat energy generated has a sensitizing effect on chemotherapy, thus increasing the concentration of chemotherapy drugs in tumor cells and meanwhile reducing the toxic and side effects of the drugs on surrounding normal cells or tissues, thereby achieving the dual anti-tumor effects of thermal therapy and chemotherapy.

Since the development of nanotechnology, there are only a handful of nano-drugs that have been approved by the FDA (Food and Drug Administration) for a clinical cancer treatment, mainly including doxorubicin (DOX) liposomes and irinotecan liposomes. Nowadays, more and more drug-loading nanoparticle composite systems are being developed and researched in the laboratory, although still far from clinical trials. In this process, in vivo animal experiments are particularly important, which may provide an indispensable research basis for clinical trials.

### SUMMARY

In view of the problems above, an objective of the present disclosure is to provide a graphene oxide-based composite nanoparticle drug delivery system. In the present disclosure, a general idea is to firstly study the photothermal stability and the in vitro photothermal efficacy of the constructed composite nanoparticle drug delivery system under the photothermal effect, then to determine the safety of the drug delivery system, and finally to research the in vivo anti-tumor effect in nude mice.

The present disclosure further provides a method for preparing the graphene oxide-based composite nanoparticle drug delivery system.

In order to achieve the objective above, the present disclosure is implemented according to the following technical solutions.

The present disclosure provides a graphene oxide-based composite nanoparticle drug delivery system, raw materials for preparing the composite nanoparticle drug delivery system comprise: an aptamer NH₂-AS1411, monolayer graphene oxide (GO), chitosan oligosaccharide (CO), γ-polyglutamic acid (γ-PGA), 1-(3-dimethylaminopropyl)-3-ethylcarbodiimide (EDC), N-hydroxysuccinimide (NHS), a 2-morpholino ethanesulfonic acid buffer (MES buffer) and a phosphate buffer solution buffer (PBS buffer).

The present disclosure further provides a method for preparing the graphene oxide-based composite nanoparticle drug delivery system, including the following steps:

(1) dissolving monolayer GO in ultrapure water and pulverizing under sonication, and then centrifuging a resultant solution after the sonication to remove unexfoliated GO to obtain a GO suspension;

(2) adjusting a pH value of the GO suspension to be within a range 5 to 6 with the MES buffer, and adding EDC and NHS in sequence to obtain a mixture; then sealing the mixture after sonication and placing on a shaker for reacting, followed by centrifuging to remove a supernatant to obtained a GO precipitate;

(3) dissolving CO in the PBS buffer under sonication to obtain a CO solution; resuspending the GO precipitate with the CO solution, adjusting a pH value of a resultant mixed suspension to be within a range of 7.2 to 7.5 with the PBS buffer, then sealing after sonication and placing on a shaker for reacting; subsequently, centrifuging and washing a resultant reaction solution and then dialyzing to obtain GO-CO;

(4) dissolving γ-PGA in ultrapure water to obtain an aqueous γ-PGA solution; adjusting a pH value of the aqueous γ-PGA solution to be within a range of 5 to 6 with the MES buffer, adding EDC and NHS, and then sealing after sonication and placing on a shaker for reacting to obtain a γ-PGA solution;

(5) dissolving GO-CO in ultrapure water to obtain a GO-CO solution after sonication; adding the activated γ-PGA solution to the GO-CO solution, adjusting a pH value of a resultant mixed solution to be within a range of 7.2 to 7.5 with the PBS buffer, and then sealing after sonication and placing on a shaker for reacting; thereafter, centrifuging and washing a resultant reaction solution, followed by dialyzing and freeze-drying to obtain a GO-CO-γ-PGA powder; and

(6) adding GO-CO-γ-PGA to a 20 mM Tris-HCl buffer containing 0.1M KCl and dissolving under sonication to obtain a GO-CO-γ-PGA solution; adding EDC and NHS in sequence, placing on a shaker after sonication for activation, and then adding NH₂-AS1411 for reacting, followed by centrifuging, washing and dialyzing to obtain APT-GO-CO-γ-PGA.

In some embodiments, in step (1), a concentration of GO in ultrapure water is 4 mg/mL; the pulverization under sonication is conducted at a power of 598 W for 1 h; the centrifugation is conducted at 8,000 r/min for 40 min.

In some embodiments, in step (2), a concentration of EDC in the GO suspension is 8 mg/mL; a mass ratio of EDC to NHS is 2:3; the reaction is conducted at 37° C. for 15 min; the centrifugation is conducted at 12,000 r/min for 10 min.

In some embodiments, in step (3), a concentration of the CO solution is 20 mg/mL; a mass ratio of CO to GO is 5:1; the reaction is conducted at 37° C. for 10 h.

In some embodiments, in step (4), the aqueous γ-PGA solution has a concentration of 2 mg/mL; a mass ratio of γ-PGA to EDC is 5:48; a mass ratio of EDC to NHS is 8:5; the reaction is conducted at 37° C. for 15 min.

In some embodiments, in step (5), a concentration of the GO-CO solution is 4 mg/mL; a mass ratio of GO-CO to γ-PGA is 2:1; the reaction is conducted at 37° C. for 10 h.

In some embodiments, in step (6), the GO-CO-γ-PGA solution has a concentration of 1 mg/mL; the Tris-HCl buffer has a pH value of 7.4; a mass ratio of GO-CO-γ-PGA to EDC is 5:212; a mass ratio of EDC to NHS is 106:159; the activation is conducted at 37° C. for 15 min; a concentration of NH₂-AS1411 is 100 μM; a ratio of GO-CO-γ-PGA to NH₂-AS1411 is 1 mg:1 mL; the reaction is conducted for 10 h to 12 h

The present disclosure further provides a use of the graphene oxide-based nanoparticle drug delivery system in loading with doxorubicin (DOX) to prepare a drug for cervical cancer and to act as a photothermal agent for tumor ablation, wherein when the composite nanoparticle drug delivery system is used to prepare the drug for cervical cancer for application, an intensity of a near-infrared laser irradiation at 808 nm required is 2 W/cm².

The present disclosure has the following beneficial effects:

(1) The present disclosure provides a novel drug-loading nanoparticle system. The research results of photothermal performance show that the drug-loading nanoparticle system APT-GO-CO-γ-PGA is more sensitive to light, and its temperature change exhibits solution concentration dependence and near-infrared irradiation intensity dependence. In vitro photothermal experiment demonstrates that the drug-loading nanoparticle system may be used as a photothermal agent for tumor ablation.

(2) The drug delivery system prepared in the present invention can actively accumulate at a tumor site and stimulate drug release to further destroy tumor tissues. Differences in outcome are attributed to the superiority of synergistic therapy over a monotherapy. Cytotoxicity experiments reveal that the prepared APT-GO-CO-γ-PGA has a function of synergistic chemical therapy and photothermal therapy.

(3) The novel drug-loading nanoparticle system of the present invention demonstrates via erythrocyte hemolysis experiments that the graphene oxide nanocomposite has a hemolysis rate of less than 5%, indicating a desirable biocompatibility as a biological carrier. It is demonstrated at the animal level that APT-GO-CO-γ-PGA-DOX has a cervical cancer targeting, and shows the most significant treatment effect on cervical cancer under a near-infrared laser irradiation of 2 W/cm². These results have a positive significance for promoting the application of drug-loading nanoparticle composite systems based on graphene oxide in cancer treatment.

(4) The nanoparticle drug delivery system prepared in the present invention has no obvious toxic or side effects on normal tissues and organs of organisms, and thus may minimize damages to non-tumor sites and plays a role in tumor treatment in vivo safely and efficiently. A valuable reference method is provided for constructing a targeting drug-loading nanoparticle system for clinical cancer treatment.

### BRIEF DESCRIPTION OF THE DRAWINGS

FIG. 1 shows a temperature change of different materials at 1 mg/ml under irradiation of a near-infrared laser (808 nm, 1.5 W/cm²);

FIG. 2 shows a temperature change of APT-GO-CO-γ-PGA at 1 mg/ml under different laser intensities;

FIG. 3 shows a temperature change of APT-GO-CO-γ-PGA under different concentrations with the same irradiation intensity of a near-infrared laser (808 nm, 2 W/cm²);

FIG. 4 shows a cycle diagram of heating and cooling five times;

FIG. 5 shows a cell viability of Hela cells treated with different concentrations of APT-GO-CO-γ-PGA and APT-GO-CO-γ-PGA-DOX under laser irradiation (2 W/cm², 5 min) for 24 h (error bars are obtained from four independent measurements);

FIG. 6 shows a diagram of an erythrocyte hemolysis experiment;

FIG. 7 shows an in vivo imaging diagram;

FIG. 8 shows a fluorescent image of major organs 12 h after injection;

FIG. 9 shows in vivo accumulation 12 h after injection;

FIG. 10 shows a diagram of body weight change of nude mice after treatment;

FIG. 11 shows a diagram of effect of different treatment methods on a tumor volume;

FIG. 12 shows an image of effect of different treatment methods on a tumor volume; and

FIG. 13 shows H&E staining of major organs and tumors with different treatments.

### DETAILED DESCRIPTION OF THE EMBODIMENTS

The technical solutions of the present disclosure are further explained and illustrated below by way of specific examples.

### Example 1

(1) 80 mg of monolayer GO was dissolved in 20 mL of ultrapure water and pulverized with an ultrasonic cell disruptor at 598 W for 1 h.

(2) The solution obtained after sonication was centrifuged at a speed of 8,000 r/min for 40 min to remove unexfoliated GO to obtain a GO suspension.

(3) 20 mL (4 mg/mL) of the GO suspension was adjusted to a pH value within a range of 5-6 with a MES buffer, 160 mg of EDC and 240 mg of NHS were added in sequence, and the mixture obtained was sonicated for 10 min. Subsequently, the mixture was sealed and placed on a shaker for reacting at 37° C. for 15 min. The reaction solution thus obtained was centrifuged in a centrifuge at 12,000 r/min for 10 min and a supernatant was removed.

(4) 400 mg of CO was dissolved in 20 mL of a PBS buffer, followed by sonication for 10 min for a full dissolution.

(5) The GO precipitate obtained above was resuspended with the CO solution. The mixed suspension was adjusted to a pH value within a range of 7.2 to 7.5 with a PBS buffer, followed by sonication for 10 min. Subsequently, the mixed suspension was sealed and placed on a shaker for reacting at 37° C. for 10 h. Thereafter, the reaction solution thus obtained was centrifuged and washed, and was dialyzed with a dialysis bag (Mw=8-14 KDa) for 2 d.

(6) 20 mg of γ-PGA was dissolved in 10 mL of ultrapure water and then adjusted to a pH value within a range of 5 to 6 with a MES buffer. 192 mg of EDC and 120 mg of NHS were added, followed by sonication for 10 min. Subsequently, the mixed solution obtained was sealed and placed on a shaker for reacting at 37° C. for 15 min.

(7) 40 mg of GO-CO obtained above was dissolved in 10 mL of ultrapure water, followed by sonication for 10 min. The activated γ-PGA solution was added to the GO-CO solution and adjusted to a pH value within a range of 7.2 to 7.5 with a PBS buffer, followed by sonication for 10 min. Subsequently, the mixed solution thus obtained was sealed and placed on a shaker for reacting at 37° C. for 10 h. Thereafter, the reaction solution obtained was centrifuged and washed, and dialyzed with a dialysis bag (Mw=8-14 KDa) for 2 d, followed by freeze-drying to obtain a GO-CO-γ-PGA powder.

(8) 10 mg of GO-CO-γ-PGA was dissolved in 10 mL of a 20 mM Tris-HCl buffer (pH 7.4) containing 0.1 M KCl under sonication, 424 mg of EDC and 636 mg of NHS were then added in sequence, followed by sonication for 10 min. Subsequently, the mixed solution thus obtained was placed on a shaker for activating at 37° C. for 15 min. 10 μL of NH₂-AS1411 (100 μM) was added thereto for reacting for 10 h to 12 h. Thereafter, the reaction solution obtained was centrifuged and washed, and was dialyzed with a dialysis bag (Mw=8-14 KDa) for 2 d to obtain an APT-GO-CO-γ-PGA solution.

(9) 10 mL of a DOX solution with a concentration of 1.5 mg/mL was added to the APT-GO-CO-γ-PGA solution above, and the mixture obtained was allowed to react under stirring for 24 h at room temperature in the dark. The unloaded drugs were removed by centrifugation on a high-speed centrifuge.

Results and Discussion:

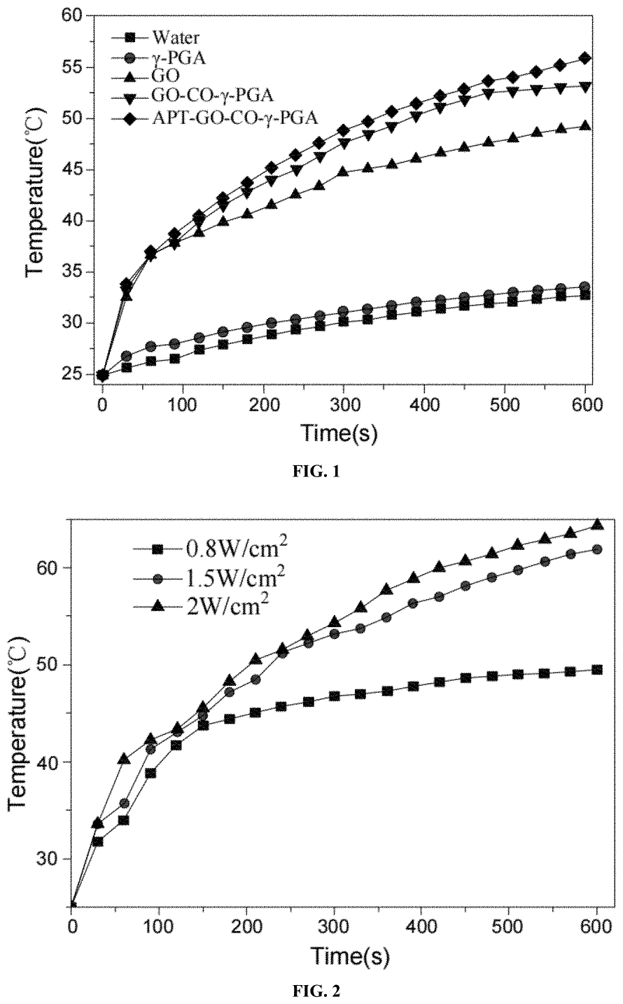

1. The photothermal effects of the nano-system were studied as to the temperature changes with different materials, different concentrations of APT-GO-CO-γ-PGA and different powers of laser irradiation at 808 nm at the same concentration. It may be seen from FIG. 1 to FIG. 3 that under the irradiation of a near-infrared laser (808 nm, 1.5 W/cm²), among the different materials, the APT-GO-CO-γ-PGA is more sensitive to light, and the temperature changes increase with an increase in material concentration and light intensity. A 1 mg/ml APT-GO-CO-γ-PGA solution was irradiated for 10 min using a near-infrared laser at 808 nm (2 W/cm²), and then the laser irradiation was removed and the solution was cooled for 20 min to return to initial temperature, during which a record was made every 30 s. The process above was repeated five times to end the experiment. It may be seen from FIG. 4 that the experimental results are basically the same and the change in temperature is also maintained at the same level, indicating that APT-GO-CO-γ-PGA has stability under light.

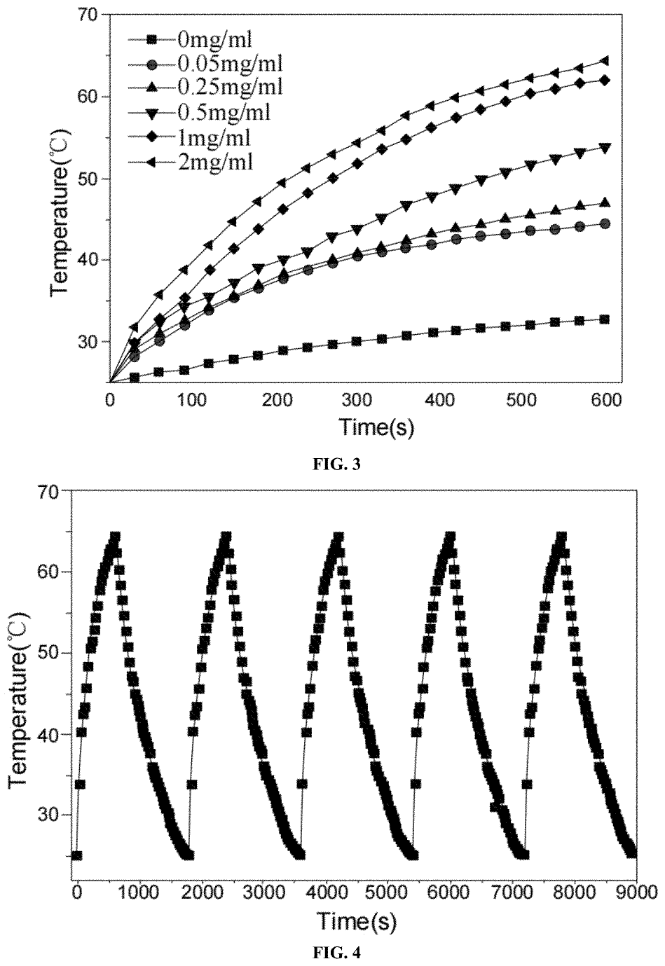

2. To study the photothermal efficacy of the drug-loading nanocomposites in vitro, a blank group, a nanoparticle carrier group, a free drug group, a drug-loading nanoparticle group and a corresponding photothermal group were set according to experimental needs. 100 μL of a cell suspension was added to each well of a sterile 96-well plate, and then incubated in an incubator at 37° C. for 24 h. Different concentrations of APT-GO-CO-γ-PGA and APT-GO-CO-γ-PGA-DOX were added thereto, respectively, and incubated in an incubator at 37° C. for another 24 h. The photothermal group was subjected to a near-infrared irradiation at 808 nm (2 W/cm², 5 min) after incubation for 3 h. The cell viability was detected by an MTT assay. It may be seen from FIG. 2 and FIG. 3 that APT-GO-CO-γ-PGA is more sensitive to light and exhibits the solution concentration dependence and the near-infrared irradiation intensity dependence in temperature change.

3. Establishment of models of heteroplastic tumors in nude mice with human cervical cancer cells Hela

15 female 3-week-old BALB/c SPF-grade nude mice were injected with human cervical cancer cells Hela in a cell injection dose of 5×10⁷ cells/mouse. The cells were passaged to ensure that all the injected cells were in a logarithmic growth phase in desirable condition, and washed with PBS, digested with trypsin and collected by centrifugation. The concentration of the cells was adjusted to about 5×10⁷ cells/mL with saline, and 0.2 mL of a cell suspension was injected subcutaneously into the left anterior axilla of each nude mouse with a syringe. Visible tumor growth occurred 3 d after inoculation; after 7 d, all 15 mice developed tumors with a solid tumor volume of about 50 mm³ and a final tumor formation rate of 100%. After the tumor volume of the nude mice reached 100 mm³, drug administration was started. The nude mice were randomly divided into a saline negative control group, a free DOX group, a GO-CO-γ-PGA-DOX group, an APT-GO-CO-γ-PGA-DOX group and an APT-GO-CO-γ-PGA-DOX-NIR group, with 3 mice in each group. The nude mice were labeled with picric acid and weighed, and was administered once every 3 d with a dose of DOX at 5 mg/kg.

Control group: saline, Cage 1: DOX, Cage 2: GO-CO-γ-PGA-DOX, Cage 3: APT-GO-CO-γ-PGA-DOX and Cage 4: APT-GO-CO-γ-PGA-DOX-NIR, all of which were subjected to 808 nm irradiation at 2 W/cm² for 5 min.

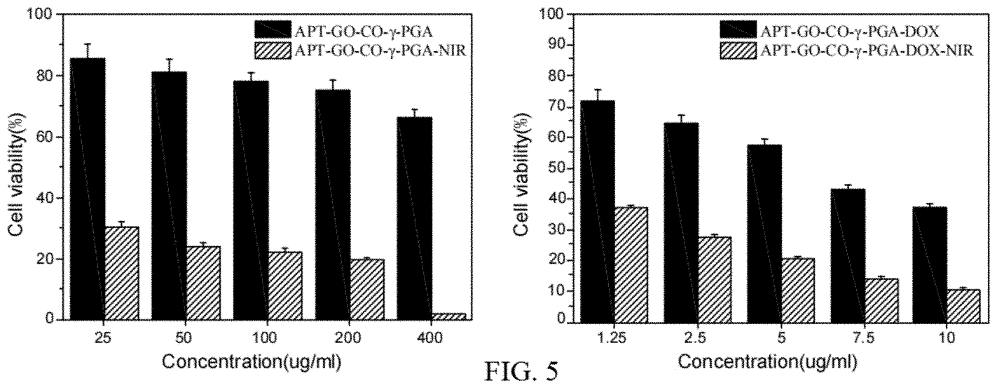

It may be seen from FIG. 5 that the experimental groups with different concentrations each have a significantly decreased cell viability after being irradiated with a near-infrared laser (808 nm, 2 W/cm²) for 5 min compared with the control group; moreover, the cell viability decreases with the gradual increase in concentration of the nano-systems. The above results all prove that the graphene oxide-based drug-loading nanoparticle system has photothermal cytotoxicity that is concentration-dependent, and the synergistic therapy is superior to the monotherapy.

4. Tracking record of growth and development of nude mice: during drug administration, the body weight, diet, water drinking and body temperature of the nude mice were observed and recorded every day; the diameter and short diameter of the tumors were measured every day to calculate the tumor volumes of the nude mice so as to plot the relative tumor volume curve. As shown in FIG. 10, APT-GO-CO-γ-PGA-DOX treatment has little effect on the body weight of the nude mice, indicating that APT-GO-CO-γ-PGA has biological safety in vivo.

5. Hemolytic toxicity experiment: an extract liquor of a target to be tested was prepared following the national standard GB/T16886.5-2011, and a hemolysis rate less than 5% indicates that the target met the requirements for hemolysis of medical materials. In the experiment, saline was used in the negative control group, sterilized double-distilled water was used in the positive control group, and free DOX and drug-loading nanocomposites were used in the experimental groups, respectively. 200 μL of an erythrocyte suspension of nude mouse was added to different groups and placed in a cell incubator at 37° C. for 2 h; then a mixed solution of each group was pipetted and centrifuged at 2,500 r/min for 5 min; 100 μL of a supernatant from each group was gently pipetted to a 96-well plate, and an absorbance value of each well at 540 nm was detected using a multifunctional microplate reader. The calculation formula of the hemolysis rate was as follows:

Hemolysis rate (HR) %=(Absorbance of experimental group−Absorbance of negative control group)/(Absorbance of positive control group−Absorbance of negative control group)×100%

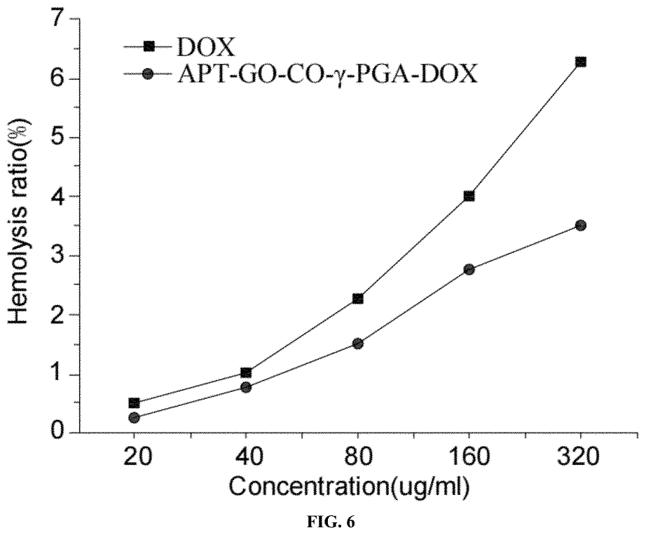

It may be seen from FIG. 6 that the graphene oxide nanocomposite has a hemolysis rate of less than 5%, indicating that APT-GO-CO-γ-PGA has desirable biocompatibility and can be used as a biological carrier.

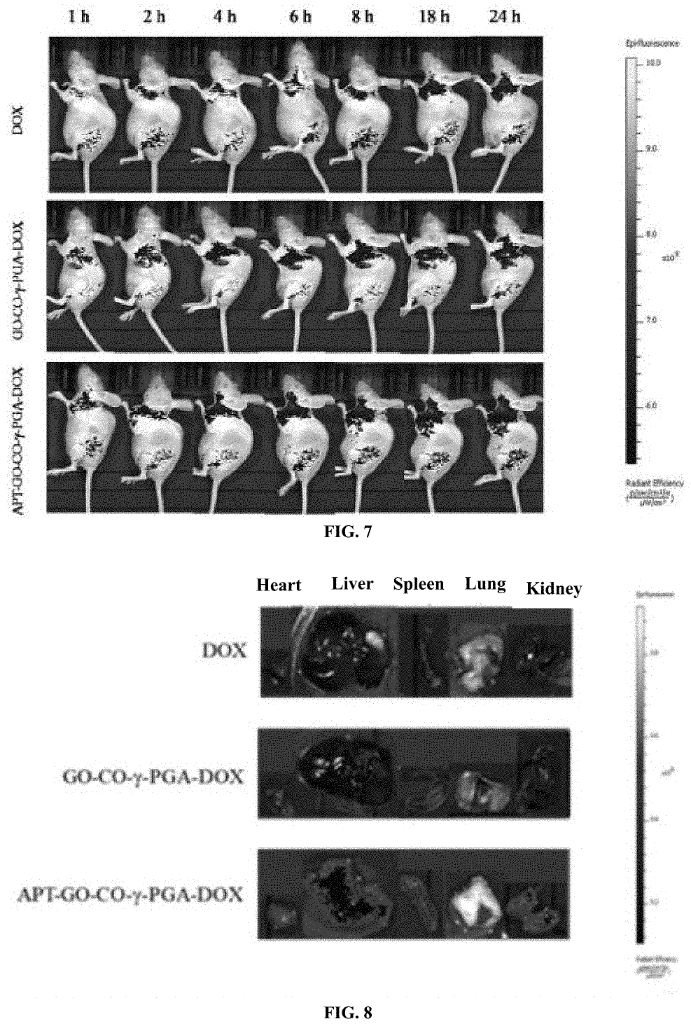

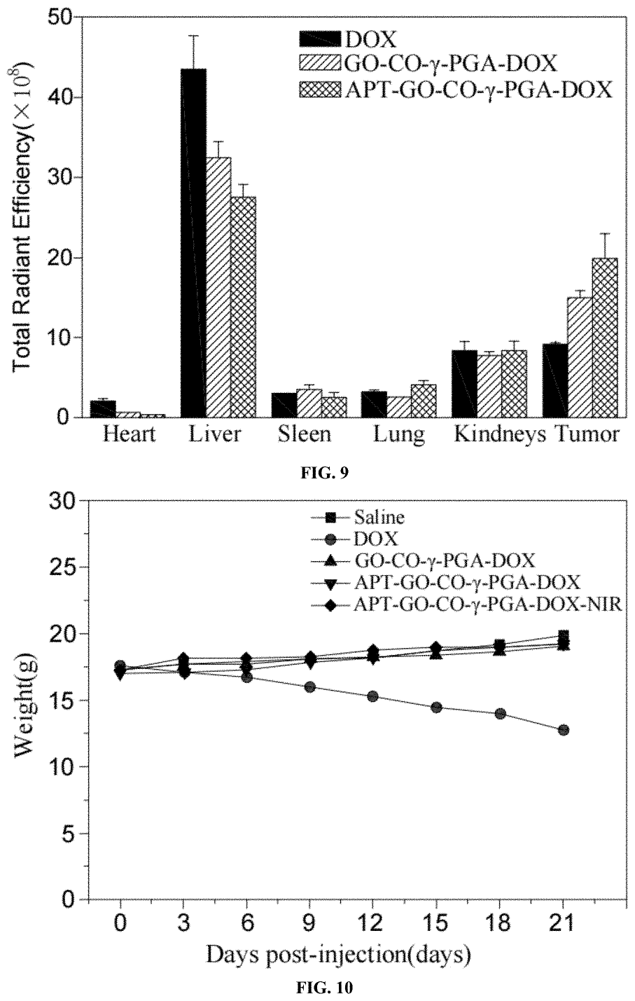

6. In vivo drug distribution and anti-tumor effect: in vivo imaging was conducted at 1 h, 2 h, 4 h, 6 h, 8 h, 18 h and 24 h after drug injection to observe the distribution and accumulation of DOX in vivo. FIG. 7 shows the in vivo imaging, and the results show that APT-GO-CO-γ-PGA has excellent targeted delivery property toward cervical cancer cells and long-term accumulation and retention properties. The mice were sacrificed 24 h after drug injection, and the heart, liver, spleen, lung, kidney and tumor were subjected to fluorescence imaging. As shown in FIG. 8 and FIG. 9, the results show that APT-GO-CO-γ-PGA-DOX reduces the toxic and side effects of the drug on surrounding normal cells or tissues.

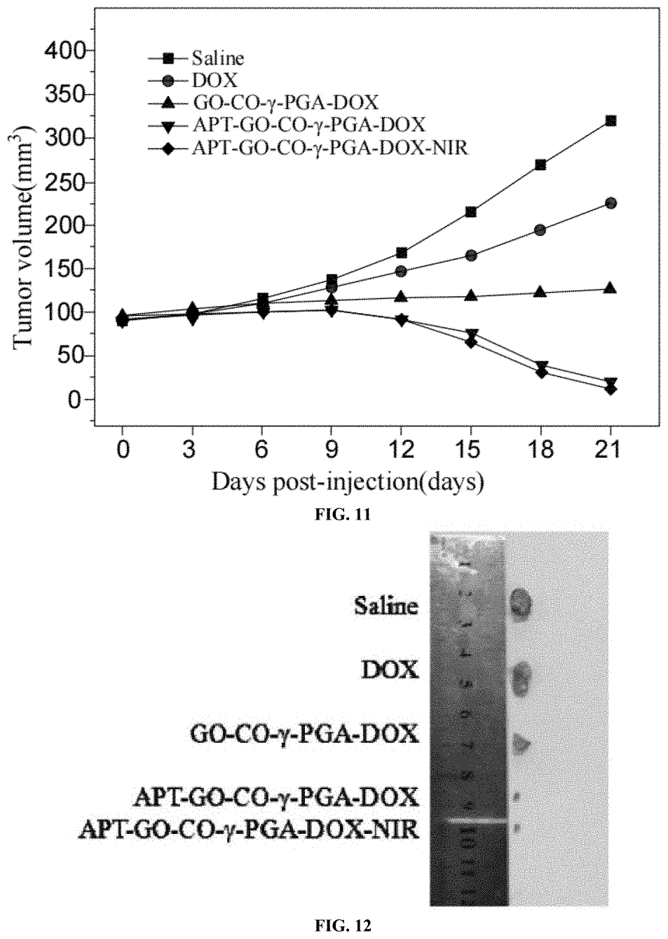

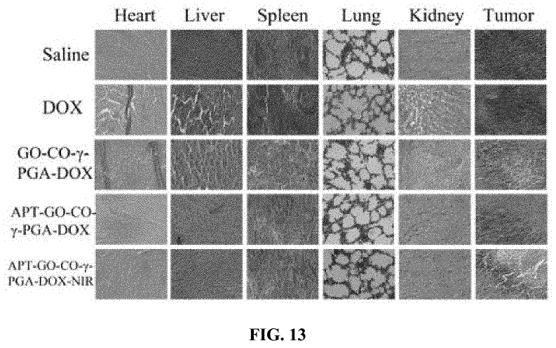

7. H&E staining: on the 21st day, 2 h after administration, the nude mice were sacrificed by neck dislocation, then the tumor, heart, liver, spleen, lung and kidney of the nude mice were collected via dissection, and the tumor volume was measured to calculate a tumor inhibition rate. FIG. 11 and FIG. 12 show the effects of different treatment methods on tumor volume, in which APT-GO-CO-γ-PGA-DOX-NIR significantly kills tumor cells, achieving dual anti-tumor effects of thermal therapy and chemotherapy. The heart, liver, spleen, lung, kidney and tumor were made into sections and subjected to hematoxylin-eosin staining for histopathological observation, as shown in FIG. 13.

When the intensity of the near-infrared laser is reduced (808 nm, 1 W/cm²), the synergistic effect of photothermal therapy and chemotherapy is not obviously observed. When the intensity of the near-infrared laser is increased (808 nm, 3 W/cm²), great damages are caused to the skin of the nude mice.

1. A graphene oxide-based composite nanoparticle drug delivery system, wherein raw materials for preparing the composite nanoparticle drug delivery system comprise: an aptamer NH₂-AS1411, monolayer graphene oxide (GO), chitosan oligosaccharide (CO), γ-polyglutamic acid (γ-PGA), 1-(3-dimethylaminopropyl)-3-ethylcarbodiimide (EDC), N-hydroxysuccinimide (NETS), a 2-morpholino ethanesulfonic acid buffer (MES buffer) and a phosphate buffer solution buffer (PBS buffer).

2. A method for preparing the graphene oxide-based composite nanoparticle drug delivery system according to claim 1, comprising:
(1) dissolving monolayer GO in ultrapure water and pulverizing under sonication, and then centrifuging a resultant solution after the sonication to remove unexfoliated GO to obtain a GO suspension;
(2) adjusting a pH value of the GO suspension to be within a range 5 to 6 with the MES buffer, and adding EDC and NETS in sequence to obtain a mixture; then sealing the mixture after sonication and placing on a shaker for reacting, followed by centrifuging to remove a supernatant to obtained a GO precipitate;
(3) dissolving CO in the PBS buffer under sonication to obtain a CO solution; resuspending the GO precipitate with the CO solution, adjusting a pH value of a resultant mixed suspension to be within a range of 7.2 to 7.5 with the PBS buffer, then sealing after sonication and placing on a shaker for reacting; subsequently, centrifuging and washing a resultant reaction solution and then dialyzing to obtain GO-CO;
(4) dissolving γ-PGA in ultrapure water to obtain an aqueous γ-PGA solution; adjusting a pH value of the aqueous γ-PGA solution to be within a range of 5 to 6 with the MES buffer, adding EDC and NETS, and then sealing after sonication and placing on a shaker for reacting to obtain a γ-PGA solution;
(5) dissolving GO-CO in ultrapure water to obtain a GO-CO solution after sonication; adding the activated γ-PGA solution to the GO-CO solution, adjusting a pH value of a resultant mixed solution to be within a range of 7.2 to 7.5 with the PBS buffer, and then sealing after sonication and placing on a shaker for reacting; thereafter, centrifuging and washing a resultant reaction solution, followed by dialyzing and freeze-drying to obtain a GO-CO-γ-PGA powder; and
(6) adding GO-CO-γ-PGA to a 20 mM Tris-HCl buffer containing 0.1M KCl and dissolving under sonication to obtain a GO-CO-γ-PGA solution; adding EDC and NHS in sequence, placing on a shaker after sonication for activation, and then adding NH₂-AS1411 for reacting, followed by centrifuging, washing and dialyzing to obtain APT-GO-CO-γ-PGA.

3. The method according to claim 2, wherein in step (1), a concentration of GO in ultrapure water is 4 mg/mL; the pulverization under sonication is conducted at a power of 598 W for 1 h; the centrifugation is conducted at 8,000 r/min for 40 min.

4. The method according to claim 2, wherein in step (2), a concentration of EDC in the GO suspension is 8 mg/mL; a mass ratio of EDC to NHS is 2:3; the reaction is conducted at 37° C. for 15 min; the centrifugation is conducted at 12,000 r/min for 10 min.

5. The method according to claim 2, wherein in step (3), a concentration of the CO solution is 20 mg/mL; a mass ratio of CO to GO is 5:1; the reaction is conducted at 37° C. for 10 h.

6. The method according to claim 2, wherein in step (4), the aqueous γ-PGA solution has a concentration of 2 mg/mL; a mass ratio of γ-PGA to EDC is 5:48; a mass ratio of EDC to NHS is 8:5; the reaction is conducted at 37° C. for 15 min.

7. The method according to claim 2, wherein in step (5), a concentration of the GO-CO solution is 4 mg/mL; a mass ratio of GO-CO to γ-PGA is 2:1; the reaction is conducted at 37° C. for 10 h.

8. The method according to claim 2, wherein in step (6), the GO-CO-γ-PGA solution has a concentration of 1 mg/mL; the Tris-HCl buffer has a pH value of 7.4; a mass ratio of GO-CO-γ-PGA to EDC is 5:212; a mass ratio of EDC to NHS is 106:159; the activation is conducted at 37° C. for 15 min; a concentration of NH₂-AS1411 is 100 μM; a ratio of GO-CO-γ-PGA to NH₂-AS1411 is 1 mg:1 mL; the reaction is conducted for 10 h to 12 h.

9. Use of the graphene oxide-based composite nanoparticle drug delivery system according to claim 1 in loading with doxorubicin (DOX) to prepare a drug for cervical cancer and to act as a photothermal agent for tumor ablation, wherein when the composite nanoparticle drug delivery system is used to prepare the drug for cervical cancer for application, an intensity of a near-infrared laser irradiation at 808 nm required is 2 W/cm².
